(19) United States
(12) Patent Application Publication
(10) Pub. No.: US 2009/0177183 A1
(43) Pub. Date: Jul. 9, 2009

(54) CELL DELIVERY CATHETERS WITH DISTAL TIP HIGH FIDELITY SENSORS

(76) Inventors: Kai Pinkernell, Encinitas, CA (US); Jose Moya, Poway, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/090,241

(22) PCT Filed: Oct. 16, 2006
(86) PCT No.: PCT/US2006/040221

§ 371 Date: Sep. 10, 2008

Related U.S. Application Data
(60) Provisional application No. 60/727,174, filed on Oct. 14, 2005.

(51) Int. Cl. A61M 25/10; A61M 25/01

(52) U.S. Cl. 604/506; 604/164.13; 604/96.01

(57) ABSTRACT

The present invention relates to over the wire cell delivery catheters with high fidelity sensors at their distal end. These catheters are comprised of flow rate sensors and/or pressure transducer sensors. These catheters can also be comprised of occlusion balloons. The catheters of the present invention allow for administration of the highest and safest dose of therapeutic agents, e.g., adipose derived regenerative cells, on an individualized basis.

### BACKGROUND OF THE INVENTION

Optimizing delivery of therapeutic cells and other agents to a site of injury is an ongoing and active area of investigation. Certain therapeutic agents, such as regenerative cells (including stem cells and progenitor cells) are ineffectual unless they can reach the site of injury. Towards this end, several modes of delivery have been investigated, including direct intramuscular injection, intravenous administration and intravascular injection.

Direct intramuscular injection is advantageous in that the therapeutic agents can be directly delivered to the damaged area. However, this method often requires a surgical procedure that allows direct visualization of the affected organ or area, which can be time-consuming, particularly in the clinical setting. It has, however, been used with various cell types in both basic biomedical research and in the clinical setting with beneficial effects.

Intravenous administration of cells is the easiest mode of delivery. However, it also suffers from certain disadvantages, including a possible entrapment of the cells (particulates) in the capillary system of the lungs, the dilution through pooling with venous blood, the length of time that it takes for the therapeutic agents to migrate to the site of the injury and the ability of the certain therapeutic agents such as cells to survive during this time frame. Furthermore, the extent that cells (e.g., regenerative cells) are able to home to the site of injury is still under investigation.

Another mode of delivery is the intraarterial approach. In this technique, therapeutic agents, e.g., cells, are delivered via an infusion catheter or an over-the-wire balloon catheter. This mode of delivery appears to be superior to intramuscular or intravenous administration because it allows for a more even distribution of cells throughout the affected region which results in a higher precision therapy.

None of the prior art delivery mechanisms, however, have the ability to determine the highest and safest dose of therapeutic agents without adversely affecting blood flow and further compromising organ function.

### SUMMARY OF THE INVENTION

The present invention provides a high-fidelity cell delivery catheter that allows the physician to administer the highest and safest dose of a therapeutic or diagnostic agent via the target vessel. The catheter is comprised of a high-fidelity sensor at the distal tip. The sensor is, for example, a pressure sensor or a flow rate sensor. An increase in pressure in the target vessel under balloon inflation can signal occlusion of the target vessel by the therapeutic agent. Also, a decrease in flow rate can signal occlusion of the target vessel by the therapeutic agent. Occlusion of the target vessel by the agent is undesirable as it results in the loss of blood flow to areas served by the target vessel and further compromises organ function. By monitoring the change in pressure and/or flow rate, the physician can accurately determine when the target vessel and its capillary system is approaching capacity for the therapeutic agent. The physician can halt further infusion of the therapeutic agent prior to the target vessel reaching capacity. This prevents occlusion of the target vessel and provides the highest and safest dose of therapeutic agent that a particular patient can accommodate. This approach allows a tailored dosing of the agent, since the size of the target vessel and therefore the affected tissue (since the artery size is proportional to the tissue amount and capillary count) is different in each patient.

Accordingly, the present invention provides a therapeutic agent delivery catheter comprising an elongated portion, a proximal end, a distal end, a guide wire, one or more lumens, and one or more sensors at the distal tip. In one embodiment, the sensor is a flow rate sensor. In a particular embodiment, the therapeutic agent is adipose derived regenerative cells. The therapeutic agent, e.g., adipose derived regenerative cells, can be used in intracoronary applications. In embodiments it can be used in applications relating to the brain, kidney, liver, pancreas, and certain other parts of the body. The sensor used is a high fidelity sensor. In certain embodiments, wherein the distal tip of the catheter is curved. In other embodiments, the distal tip is straight.

The present invention also provides a therapeutic agent delivery catheter comprising an elongated portion, a proximal end, a distal end, a guide wire, one or more lumens, an occlusion balloon at the distal tip and a sensor distal to the occlusion balloon. In one embodiment, the sensor is a pressure transducer sensor. In a particular embodiment, the therapeutic agent is adipose derived regenerative cells. In a preferred embodiment, the adipose derived regenerative cells will be used in intracoronary applications. The figures show examples of catheters of the invention, including catheters not having an occlusion balloon.

The present invention also encompasses methods of administering a therapeutic agent comprising monitoring the flow rate or the pressure in a target vessel. Levels of flow and pressure allow administration of the highest and safest dose of therapeutic agent. In particular, administration of the therapeutic agent is halted when the flow rate decreases. Administration of the therapeutic agent is also halted when the pressure increases.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description.

### BRIEF DESCRIPTION OF THE DRAWINGS

FIG. 1. A pressure transducer sensor cell delivery balloon catheter. The catheter 10 comprises a protruding occlusion balloon 15, a balloon inflation lumen 11, a guide wire and cell delivery lumen 12, a pressure sensor or flow transducer 13, 18, and a control box 17.

FIG. 2. A pressure transducer sensor cell delivery catheter with a straight tip at its distal end. This drawing shows an infusion catheter having a pressure sensor or flow transducer at its distal tip. Sensors can be present at any location at the distal tip of the catheter, e.g., as described in FIG. 3.

FIG. 3. A pressure transducer sensor cell delivery catheter with a curved tip at its distal end. This drawing illustrates location options, e.g., locations for side-mounted sensors, on a catheter having a curved tip.

FIG. 4. A flow rate sensor cell delivery catheter with a curved tip at its distal end. In this system, the catheter 10 can have flow rate sensors 14, e.g., thermodilation or doppler sensors, at various positions at its distal tip, as indicated in the diagram. The catheter flow sensor lumens 11 can accommodate flow sensor wires. The flow rate sensors can also be present on wires extending from any position at the distal tip of the catheter. The catheter further comprises a cell delivery and guide wire lumen 12. Connectors 16 can be used to connect the lumens to the appropriate instrumentation, e.g., the flow sensor lumen can be connected to a control box 17, for processing information collected. In embodiments, sensors that measure parameters other than flow rate can be so positioned. The lumens 11 would thus be used for connection to any instrumentation appropriate for the particular sensors used.

FIG. 5. A flow rate sensor cell delivery catheter with a straight tip at its distal end. The catheter 10 has a flow rate sensor 14 distal to its tip. Alternatively, the flow rate sensor can be present at any position at the distal tip, e.g., as shown in FIG. 6. The flow rate sensors can be present on wires extending from any point on the distal tip of the catheter. The catheter also comprises a cell delivery and guide wire lumen 12.

FIG. 6. A flow rate sensor cell delivery catheter with a straight tip at its distal end—2. This drawing shows another example of a catheter as described by FIG. 5. Here, the flow rate sensor 14 is present in a different position at the distal end of the catheter tip.

FIG. 7. A flow rate sensor cell delivery balloon catheter with a straight tip at its distal end and a balloon proximal to the flow rate sensor. This drawing depicts a system comprising a catheter having an occlusion balloon 15 proximal to the flow rate sensor 14. The flow rate sensor is distal to the tip of the catheter. Alternatively, the flow rate sensors can be present at any position at the distal tip, e.g., as shown in FIG. 8. The flow rate sensors can be present on wires extending from any point at the distal tip of the catheter. A balloon inflation lumen 11 allows the balloon to be inflated and deflated as needed.

FIG. 8. A flow rate sensor cell delivery balloon catheter with a straight tip at its distal end and balloon proximal to flow rate sensor—2. This drawing shows another example of a catheter as described by FIG. 7. Here, the flow rate sensor 14 is located in a different position at the distal tip.

FIG. 9. A flow rate sensor cell delivery balloon catheter with a straight tip at its distal end and balloon distal to flow rate sensor. This drawing shows a catheter having a flow rate sensor at the distal tip but proximal to the occlusion balloon. In this system, flow rate could be measured after deflation of the balloon. In embodiments, the balloon protrudes from the tip of the catheter, as described in FIG. 1.

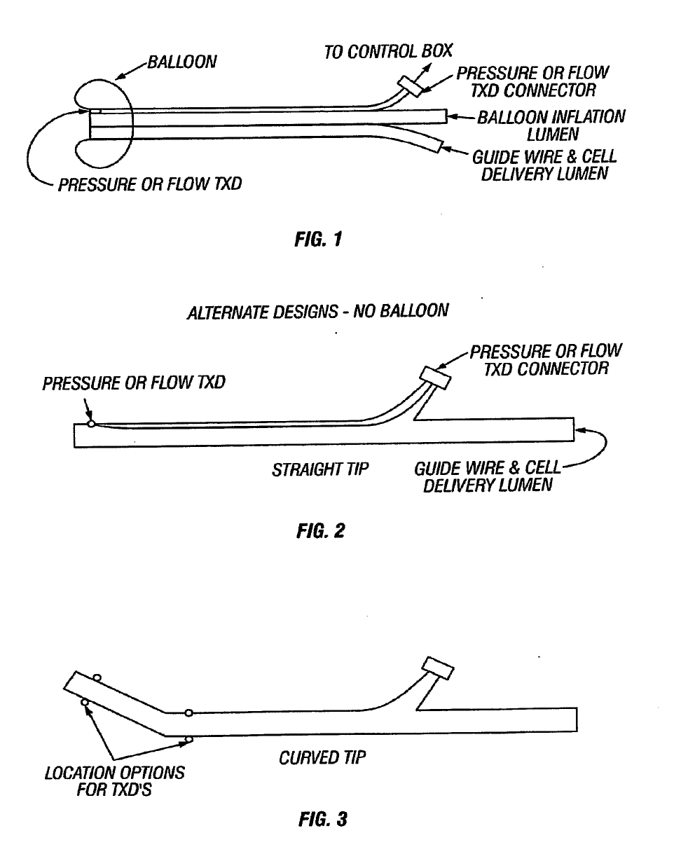

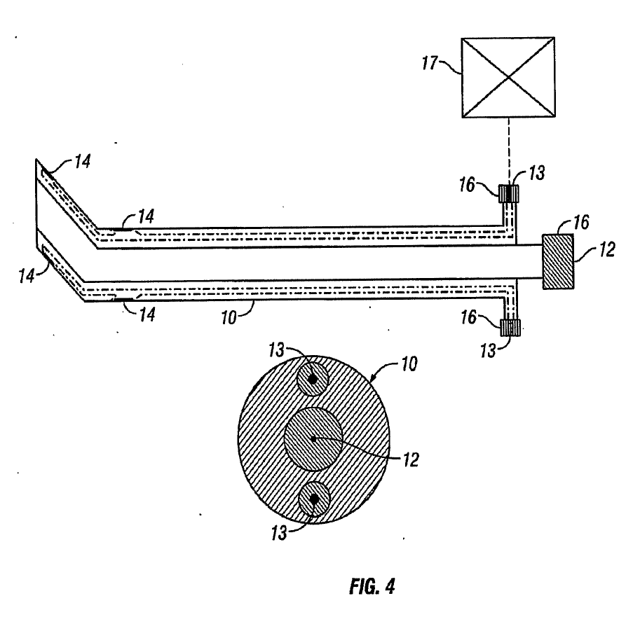

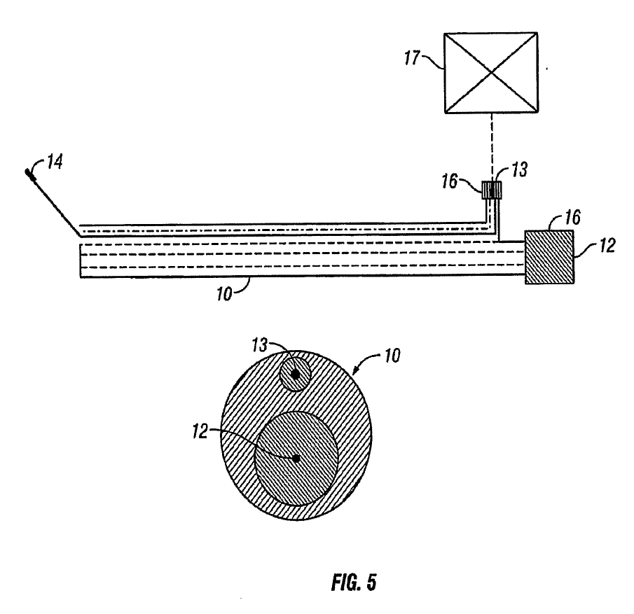

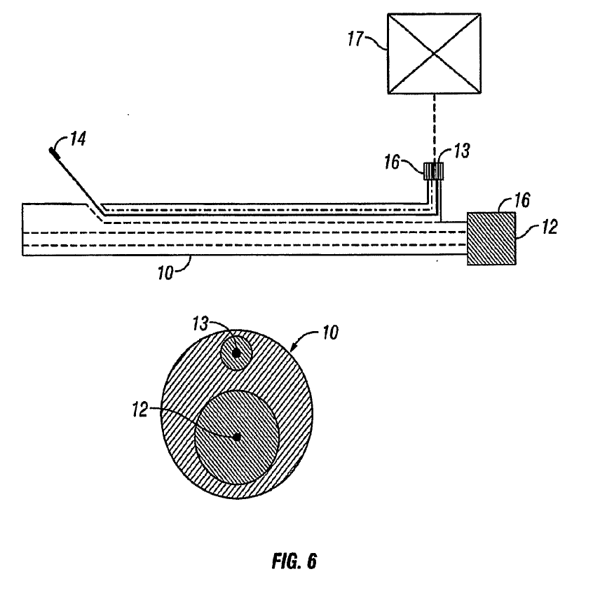

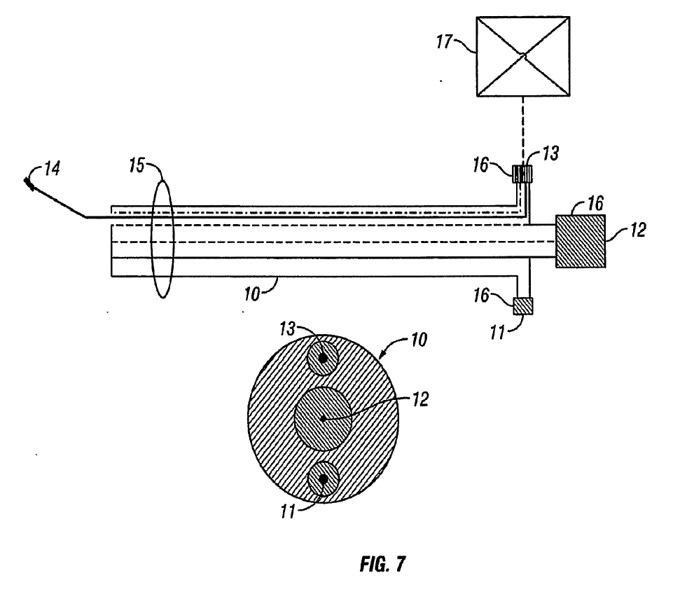

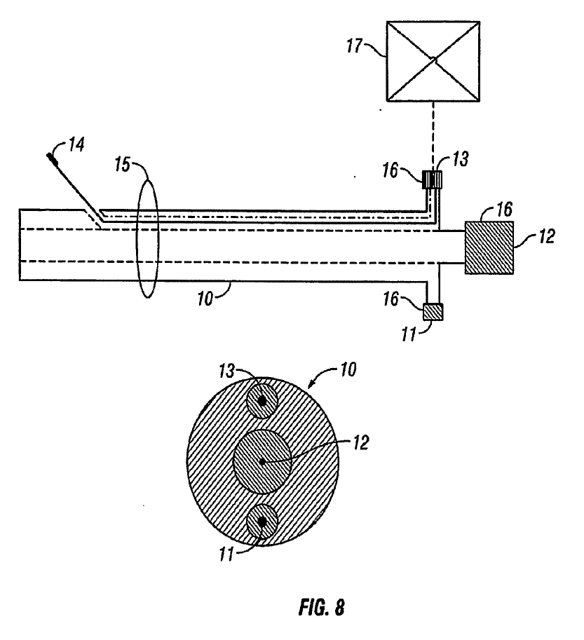

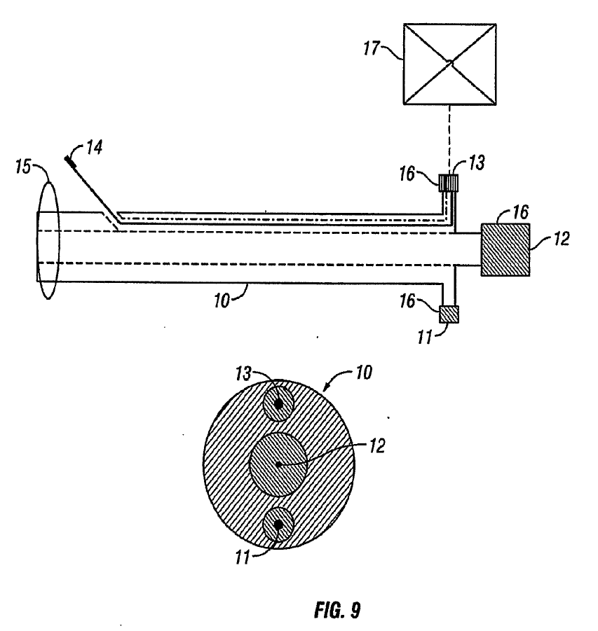

### DETAILED DESCRIPTION OF THE INVENTION

The present invention relates to a high-fidelity catheter system for delivering therapeutic or diagnostic agents, e.g., cells, to a site of injury in the body. Specifically, the catheter system includes an over-the wire catheter with at least one sensor at the distal tip or in close proximity to the distal tip (e.g., about 10, 20, 30 or 40 mm from the distal tip). In one embodiment, the sensor is a flow rate sensor. In another embodiment, the sensor is pressure-transducer sensor. In yet other embodiments, the catheter is further comprised of an occlusion balloon. In other embodiments, the catheter is comprised of multiple sensors. The catheter may also be comprised of a guide wire. The guidewire may be a steerable conventional guidewire that is movable between various positions. Various types of guidewires can be used in the catheter system of the invention. For example, a flexible, wire-like metal member having a diameter of about 0.010 to about 0.020 inches could be used. In one preferred embodiment, the cell delivery catheter is a balloon catheter comprised of a pressure transducer sensor at the distal tip or in close proximity to the distal tip. The catheter system of the present invention may also include an energy transmission means (e.g., an optical fiber) which carries energy along one or more lumens of the catheter. The energy transmission means, e.g., an optical fiber, could be used to, for example, monitor the viability of the therapeutic agent being delivered. The catheter system of the present invention may also include electronic means which could be operably linked to catheter to provide guidance data to the user of the system and permit safe navigation of the catheter. The catheter of the present invention may also include a magnetic guidance system to provide guidance to the user of the system to reach the precise spot where treatment is needed.

The catheters of the present invention are an improvement over prior art catheters in that they can monitor pressure and/or flow rate and indicate microvascular obstruction. This allows for individualized administration of the highest and safest dose of cells and/or other therapeutic or diagnostic agents. The catheter system of the present invention can be used to for a variety of diseases or disorders that require administration of therapeutic or diagnostic agents at or near the site of injury. For example, the present catheter system can be used in the brain, kidney, liver, pancreas, heart and certain other parts of the body, e.g., the urinary tract. The catheters of the present invention can be used in small animals such as mice, large animals such as dogs and sheep as well as humans.

Sensors are devices that detect physical, chemical, and biological signals and provide a way for those signals to be measured and recorded. Physical properties that can be sensed include temperature, pressure, vibration, sound level, light intensity, load or weight, flow rate of gases and liquids, amplitude of magnetic and electronic fields, and concentrations of many substances in gaseous, liquid, or solid form. Tactile sensors, typically piezoelectric materials, generate voltage when touched, squeezed, or bent, or when their temperature is changed. Other sensors can detect specific chemical pressures and fluid levels. The present invention encompasses, but is not limited to, any of the above listed types of sensors. The sensors used in the present invention will preferably emit a signal that can be read at the point of determination or transferred by wire or wireless transmission to remote locations, e.g., the proximal end of the cell delivery catheter. Preferably, the sensors used in the present invention are “smart sensors” that unite sensing capability and data processing in a single integrated circuit chip. In preferred embodiments, the sensors are embedded at the distal tip of the cell delivery catheters of the present invention. Placement of the sensor at the distal tip provides a high fidelity signal because the signal, e.g., pressure and/or flow rate, is measured directly at the source and is not subject to movement artifacts and signal fluctuations.

Flow rate sensors function by measuring blood velocity in a target vessel. Blood velocity is the quantity of blood that passes through the arterial or venous circulation within a period of time. Upon occlusion of the target vessel, the flow rate or blood velocity decreases over time. The flow rate signal expressed in graphical or tabular or other illustrative format allows the physician to determine the early signs of occlusion and generally predict the point of total occlusion. The physician can thus stop infusion at the point right before critical occlusion is reached thereby delivering the safest and highest dose of therapeutic agent on an individual basis. Critical occlusion can be defined as the amount of reduction in blood perfusion caused by the agent that will result in tissue damage. It can be particularly useful to measure flow rate during infusion in the absence of an occlusion balloon. In the absence of a balloon, blood can freely flow until infusion of an agent causes an occlusion downstream. However, blood velocity measurements are subject to errors due to non-linearity, time delay, motion artifact, bend effect, temperature effect, electrical interference and/or drift with time. To obtain a high fidelity flow rate signal, the effects of these errors must be minimized by careful design of the catheter and its sensors.

Pressure transducer sensors measure pressure. Blood flow in the body is driven by differences in pressures along a vessel (i.e., arterial and venous pressure). Upon occlusion of, e.g., a coronary artery, the pressure that can be measured distal (further down the blood stream) of the occlusion will equal the capillary pressure that is usually around 20 mmHg. Upon infusion of particulates, e.g. cells or other therapeutic agents, especially when their diameter exceeds the diameter required to pass the capillary system (usually above 8-10 μm) the capillaries will gradually become obstructed leading to an increase in resistance. The concentration of the infused therapeutic agent may also play an important role in that the higher the agent is concentrated the faster a microvascular obstruction can be observed. If the infusion is continued, i.e., if a constant flow infusion is used, and especially when back flow (due to balloon inflation) is prevented, the pressure measured distal of the balloon will gradually increase. If infusion into the capillary system of the obstructing agent is not stopped, blood flow will not resume after deflation of the balloon with detrimental effects on the affected tissue. The catheter of the present invention comprising a pressure transducer sensor allows the physician to measure pressure distal to the balloon to prevent critical obstruction of the capillary bed. However, to obtain a high fidelity pressure signal that can be used to evaluate critical microvascular obstruction, the catheter and its sensors must be carefully designed to minimize errors.

A pressure sensor within the meaning of the present invention may include any type of sensor capable of fitting on the catheter and of measuring blood pressure and producing a signal with a frequency response above approximately 15 Hz. Such sensors include but are not limited to micromanometers such as those produced by companies such as Millar, Endosonics, and Radi. These sensors typically include a small transducer exposed to arterial pressure on one side and often a reference pressure on the opposite side. Blood pressure deforms the transducer resulting in a change in resistance which is translated into a pressure reading. Alternatively, a fiber optic sensor may be used in which case pressure sensing line would comprise a fiber optic line.

Of particular importance are the characteristics of each individual sensor used with the catheters of the present invention. The important characteristics include, the frequency response, the response to rapid changes in pressure and phase delay. To accurately measure pressure in a physiological system, a frequency response from 0 to about 6 kHz is a basic requirement. A flat frequency response curve insures that the transducer is sensitive enough to detect instantaneous changes in the pressure signal arising from physiological events in a given system.

The pressure transducer sensors used in the present invention must also respond instantaneously to rapid changes in pressure. A delayed response to a pressure drop will cause distortion in the signal. Similarly, a pressure transducer should have very little to no phase delay. A phase delay is indicative of a transducer's ability to accurately reproduce a sudden change in pressure.

As set forth herein, the catheters may be comprised of more than one sensor. For example, a single sensor catheter may be a catheter with a pressure transducer sensor as described above. Alternatively, the pressure sensor may be replaced by a flow rate transducer sensor. In other embodiments, the multi-sensor catheter can be comprised of, for example, one sensor to measure pressure and another to measure flow rate. Multi-sensor catheters have been described as potentially providing better information when used in combination. (See, e.g., Pijls, et al., 2002, Circulation 105:2482-2486; Fearon, et al., 2003, Circulation 108:1605-1610, and; De Bruyne, et al., 2001, Circulation 104: 2003-2006.) Real-time, relative or absolute flow rates can be measured using a flow rate transducer sensor. For example, constant flow velocity measurements can be taken and cell delivery adjusted accordingly. The tip of the catheter may also include a bend to enhance cell delivery and flow measurements for curved or branching vessels. The flow transducer may be located proximal and/or distal to the curve to provide a location for accurate flow rate acquisition.

For single sensor catheters, the sensors may be side-mounted at the distal tip of the catheter. For dual sensor catheters, the first sensor may be side-mounted at the distal tip of the catheter and the second sensor may be mounted proximal (e.g., 2 mm-6 cm) to the first sensor or it may protrude beyond the tip. The catheter tip may be straight or curved. The catheter itself may be made of any conventional material such as polyurethane, nylon or polyurethane woven dracon. The length of the catheter can vary and can be adjusted according to the type of tissue or vessel involved. For example, in coronary intravascular applications, a catheter length in the range of about 50 cm to about 200 cm can be used. The French sizes of such catheters can range from about 2 to 8. The catheters may be reusable, repairable or disposable. The catheters can have standard connector types such as Viking connector types. In one embodiment, the catheters have electrodes in addition to pressure transducer sensors.

The cell delivery catheters of the present invention are from about 100 cm to about 250 cm in length although extensions for cables may be necessary. The end use application for the catheter will ultimately dictate the specific length. The outer diameter of the catheter may range from about 2 to about 8 French i.e. from about 0.6 mm to about 2.4 mm. The diameter can be substantially constant along the length of the catheter. The diameter of the catheter may also be larger at its proximal end and taper into a smaller diameter at its distal end. If the catheter is comprised of a guide wire, the guide wire has to be proportional to the catheter. The catheters of the present invention are designed to accommodate vessels ranging from about 2 to about 6 mm in diameter. Such vessels include, but are not limited to coronary, peripheral, renal, hepatic, ureter and gastrointestinal vessels.

The catheter of the present invention generally comprises an elongate portion having a proximal and a distal end and one or more lumens connected to said proximal end, as well as a sensor on the distal end. The catheter may further comprise an occlusion balloon. For example, a single lumen over-the wire balloon catheter utilizes the same lumen for the fluid supply/removal path, the therapeutic agent delivery path as well as the guide wire insertion path. A multi-lumen type catheter uses different lumens for guide wire insertion, therapeutic agent delivery and/or fluid supply/removal. Other lumens can be used to for example, for maintaining the blood flow between both sides of the balloon. Single, dual as well as multi-lumen balloon catheters are within the scope of the present invention. The lumen utilized for delivery of cells is one with a large diameter to minimize shear stress on the cells. In a multi-lumen balloon catheter, in addition to a lumen for a fluid supply/removal path, therapeutic cells may be administered via one lumen while another lumen can be used to infuse a secondary agent (e.g., another therapeutic agent or a diagnostic agent) if needed.

In other multi-lumen catheters, one lumen can, for example, communicate with an occlusion balloon located at the distal end of said elongate portion. Another lumen can extend the entire length of the elongate portion which allows for the placement of the catheter over a guide wire. A third lumen can also be connected to the proximal end of the elongate portion. The third lumen may be used to deliver the therapeutic agent. Any of the lumens may be adapted to receive a removable stiffening stylet to ease insertion. Each lumen may also be used for more than one purpose. For example, the same lumen may be used for placement of the guide wire and for delivery of the therapeutic agents.

In a preferred embodiment, the catheter is comprised of an elongate portion, two lumens and a sensor on the distal end. One lumen is used for placement of the guide wire and the other lumen is used for cell delivery. The sensor is a flow rate sensor. In another embodiment, the catheter is comprised of an elongate portion, two lumens, a sensor on the distal end and an occlusion balloon on the distal end. One of the lumens will be utilized for the guide wire placement (over the wire design). The same lumen will be utilized for delivery of the therapeutic agent, e.g., cells. The second lumen will be used to inflate/deflate the occlusion balloon. The sensor is a pressure transducer sensor.

The elongate portion of the catheter as well as any of the lumens may be manufactured using any of the commercially used catheter materials. For example, polyethylene, polyamide, urethane, etc. may be used. The specific material chosen will depend on the catheter's end use, the size of the target vessel, and whether or not a stylet or stylets will be used to assist during insertion and advancement. The material for the elongate portion, the lumens and the occlusion balloons may contain one or more additives such as hydrophilic coatings such as silicon, radiopaque fillers, slip additives, etc.

As set forth above, in certain embodiments, the cell delivery catheter is comprised of a pressure transducer sensor as well as an occlusion balloon. Balloon catheters have been in existence for many years and have found wide application. In general they are used whenever the occlusion of a vessel is desired such as during embolization, arteriography, preoperative occlusion, emergency control of hemorrhage, chemotherapeutic drug infusion and renal opacification procedures. For the present invention, the occlusion balloon is used to occlude the target vessel such that the therapeutic agents delivered via the catheter are concentrated and evenly distributed in the target area thereby improving therapeutic effect. The occlusion balloon of the present invention is preferably expandable up to about 5 mm to about 10 mm.

The occlusion balloon is preferably a soft, compliant balloon located at the distal tip of the catheter. The balloon is mounted onto the catheter by means that will allow the balloon to act as a soft tip without impeding the delivery of the therapeutic agent(s). This is important to prevent unwanted vessel trauma during delivery of the therapeutic agent(s), e.g., cells. Typically, the balloon is made of a moldable polymer like, for example, polyurethane, latex, silicone rubber, natural rubber, polyvinyl chloride, polyamide, polyamide elastomer, copolymer of ethylene and vinyl acetate, polyethylene, polyimide, polyethylene terephthalate, fluorine resin and the like. However, it is to be appreciated that any other resin which is flexibly extendable and shrinkable, and is harmless as a medical apparatus can also be used, as there is no special limitation on such useable materials. Additionally, the shaft may be made of any material similar to that of the balloon so long as the material used can be flexibly bent while maintaining the form of the lumen.

Either a gas or a liquid can be used as the fluid for inflating the balloon. Generally, a gas is preferable because of better safety characteristics for application of therapeutic agents. A gas gets compressed at increasing pressures. In contrast, fluids can potentially harm the vessel because a high balloon inflation pressure will rupture the vessel. However, use of a liquid is feasible and the balloon can be inflated with any suitable liquid including a physiological salt solution, a solution containing contrast medium, etc. If the balloon is inflated with gas, a gas that dissociates rapidly into the blood (in the event of an accidental rupture of the balloon) is preferred (e.g., carbon dioxide, nitrogen etc.) to avoid gas embolism in the vascular system.

The balloon catheter is comprised of a pressure transducer sensor as described above. The pressure transducer sensor is preferably located at the distal tip of the catheter and provides vessel pressure information that will be used to control both the cell delivery rate and amount. An electrical connector (such as a Viking connector) will be provided at the proximal end of the catheter for connection of the pressure transducer to an intelligent control unit. The control unit will provide automatic, closed loop, cell delivery and includes features such as automatic cut-off, alerts and alarms. The control unit will also be used to provide closed loop balloon expansion to minimize trauma to the vessel wall. The control unit will inflate the balloon just large enough to occlude blood flow and then automatically stop inflation.

In general, the shaft of a balloon catheter has high flexibility. High flexibility ensures that the shaft can flexibly curve along a bending blood vessel to smoothly guide the balloon catheter into the vessel. In some applications, however, some rigidity of the catheter shaft is required to enhance proper positioning. Thus, in certain embodiments of the pressure transducer sensor cell delivery balloon catheter further comprises a core made of metal wire or a similar material that is fixed inside the shaft.

In a manner well understood by those of skill in the art, flow rate, pressure, location of the catheter within the tissue and other parameters can be initiated, controlled and/or monitored using electronic means attached to the catheter or a system component and a conventional computer system. The interaction of the electronic means and a computer produces a signal that is displayed and periodically updated on a suitable display. The signal is monitored by the computer through a series of art-known algorithms. For example, a baseline flow rate can be measured and indicated on the display when the catheter system is initially positioned within the vessel. Upon infusion with the therapeutic agent, the flow rate can be measured periodically and indicated on the display. The flow rate measurements could be converted into a real-time visual display in the form of a graph that tracks the flow rate relative to the amount of the therapeutic agent being infused. The infusion could be continued until the graph begins to slope downward indicating that the vessel is being occluded by or is starting to be occluded by the therapeutic agent. At this point, the user of the catheter system must take steps to reduce or stop infusion as appropriate. The approximate endpoints for infusion in various tissue may be empirically determined prior use and may be incorporated into the algorithm used by the computer.

The above-described catheter systems are particularly useful in intracoronary delivery applications. Blood arrives at the heart muscle via coronary arteries which begin as vessels with a diameter of several millimeters and branch progressively to smaller and smaller vessels in order to supply all the cells of the heart muscle. Blood arriving at the heart carries oxygen and nutrients which are exchanged for carbon dioxide and other wastes produced by cellular respiration. The carbon dioxide carrying blood leaves the heart muscle via a system of coronary veins which begin as small vessels and progressively merge into larger vessels. As in other organs, the veins are approximately parallel to the arteries, although the blood flow therein is in the opposite direction. The coronary veins terminate in a reservoir referred to as the coronary sinus, which, in turn, drains into the right atrium where it mixes with venous blood from peripheral organs. Venous blood is pumped from the right atrium into the pulmonary arteries which perfuse the lung and facilitate an exchange of gases, with carbon dioxide being replaced with oxygen.

In cases where the supply of blood flowing to the heart muscle via the coronary arteries is insufficient, oxygenation of the muscle tissue of the heart is reduced, producing a condition known as cardiac ischemia. Ischemia can result in atrophy and or necrosis of tissue. In the case of cardiac ischemia, this atrophy or necrosis reduces heart function and adversely affects the blood supply to the remainder of the body. Adipose-derived regenerative cells have been shown to reverse this effect by promoting angiogenesis in the ischemic tissue and other beneficial effects. Methods of obtaining adipose derived regenerative cells are known in the art and are described in commonly owned application Ser. No. 10/877,822 entitled Systems and Methods for Separating and Concentrating Regenerative Cells from Adipose Tissue, filed on Jun. 24, 2004, which is incorporated herein in its entirety by this reference.

However, every candidate for regenerative cell therapy will have a different size of infarct and varying target vessel diameter. Using prior art catheters, the appropriate dose (in terms of efficacy and safety) could not be measured. Specifically, prior art catheters do not allow for measuring pressure or flow rate in the vessel that allows the physician to halt regenerative cell infusion and thereby prevent occlusion of the vessel. This shortcoming in the prior art prevents intravascular delivery of therapeutic agents. The present invention overcomes this limitation through the presence of a high fidelity flow rate sensor or a pressure transducer sensor at the distal tip of the catheter that informs the physician when to stop infusing the vessel with regenerative cells due to microvascular blockage.

The above-described catheter systems are also useful in treating and/or diagnosing brain and brain-related diseases and disorders. Brain diseases such as stroke and Parkinson's disease affects millions of people in the United States alone. Part of the challenge in combating these diseases is effectively delivering developing therapies inside the brain without damaging vital brain tissue in the process. Adipose tissue derived regenerative cells referred to herein can be used to treat brain diseases and disorders. Accordingly, the catheter of the present invention may be used to deliver adipose derived regenerative cells to brain tissue.

The catheter system would be appropriately modified for use in brain (or other tissues and organs) using materials and methods known to one of skill in the art. For example, the catheter would be constructed to prevent air from being trapped inside the catheter during insertion; to prevent the uneven distribution of fluids pumped into the tissue or organ during infusion therapies; to prevent damage to blood vessels and tissue from multiple insertions of the catheter; to make sure an adequate amount of therapeutic and/or diagnostic agents are delivered; and to make sure that therapeutic agents are viable when they are released. For example, to guide the catheter through the blood vessels of the brain to reach the precise spot where treatment was needed, the catheter system of the present invention would preferably include a computer that the user could use to control the guidance system and precisely direct the catheter. To prevent the problems of trapped air, the catheter could be comprised of a lumen within a lumen such that the smaller lumen is inserted within the larger lumen (e.g., both with diameters of approximately 1-2 millimeters), and leaving some space between the two lumens. This allows excess air to be forced out through the space between the lumens, rather than being forced deeper into the brain tissue. To prevent trauma when multiple insertions are required, the outer lumen can be left in place and the inner lumen can be withdrawn and reinserted without causing additional trauma to the surrounding tissue. To ensure viability of the therapeutic agent, attaching energy transmission means, e.g., optical fibers, could be attached to the lumens, to visually monitor the agent, e.g., cells.

In addition to blood vessels, the catheter system of the present invention may be used to deliver a therapeutic agent through the numerous ducts and canals in a body that transport fluids other than blood and which serve many different functions in different organs. The catheter system would be appropriately modified for use in such ducts and canals using materials and methods known to one of skill in the art. Examples include, but are not limited to, tear ducts, breast ducts, the bile duct, the ductus Wirsungii of the pancreas, vessels and ducts of the lymphatic system, the ejaculatory duct, the parotid duct, and the submaxillary duct (also referred to as Wharton's duct).

For example, delivery of therapeutic cells in breast cancer (for example, delivery of cells modified to deliver an anti-cancer agent), or repair, reconstruction, and even augmentation of the breast may be achieved by local and targeted delivery of the therapeutic cells through the breast ducts (also referred to as mammary ducts or lactiferous ducts). In this setting it may be preferable to occlude the duct behind the catheter tip to prevent cells from flowing out through the nipple. However, in another embodiment the duct is not occluded. This permits slow perfusion of a large volume of fluid through the duct. In this setting, large volumes of relatively dilute therapeutic agent populations may be delivered under physiological conditions.

Similarly, the catheter of the present invention may be passed through the alimentary canal into the intestine to the region of the pancreas and into the ductus Wirsungii. The same approaches may be used for delivery of material to the ducts of the hepatic system (the cystic duct and the common bile duct) and the lymphatic system (e.g., to deliver therapeutic or diagnostic agents to the organs served by the lymphatic system e.g., the pulmonary system and limbs).

### EXAMPLE I

### Effect of Cell Infusion on TIMI Grades with and without Balloon Occlusion

The effect of cell infusion on blood flow rate, as evaluated by TIMI grading, was evaluated. Cells were either infused during balloon occlusion or without balloon occlusion, and TIMI flow grading was used to measure and score blood flow at timepoints before, during and following the infusion.

The adipose-tissue derived cells (ADCs), prepared as described, e.g., in U. S. Pat. App. No. 20050084961, entitled “Systems and methods for separating and concentrating regenerative cells from tissue,” were administered by intracoronary infusion to a pig model of acute myocardial infarction (AMI).

For the balloon down infusion, an AMI was induced in nine animals by balloon occlusion using an angioplasty balloon and inflating it in the mid-LAD, for 3 hours. An ADC suspension with a concentration of 2.5×10⁶ cells/ml and a flow rate of approximately 1 ml/min was infused in boluses of 3 ml at the site of former balloon occlusion for AMI induction into the coronary artery using a Renegade™ microinfusion catheter (Boston Scientific). Blood flow was measured and graded by TIMI flow in each animal before occlusion, after AMI (before injection), after the 3rd injection, after the 6th injection, and at the final angiography after all injections (up to eight total) were completed (“Final”). The scores are shown in Table 1.

TABLE 1

| | | | Pre- | 3^{rd} | 6^{th} | |
|---|---|---|---|---|---|---|
| | Cell | | Injec- | Injec- | Injec- | |
| Animal | Dose | Pre-MI | tion | tion | tion | Final |
| ID | (×10⁶) | (TIMI) | (TIMI) | (TIMI) | (TIMI) | (TIMI) |
| 813 | 48.2 | 3 | 2 | 2 | 3 | 3 |
| 812 | 48.5 | 3 | 3 | 2 | 2 | 2 |
| 811 | 47 | 3 | 2 | 2 | 2 | 2 |
| 809 | 56.3 | 3 | 3 | 2 | 2 | 2 |
| 808 | 55.2 | 3 | 3 | 3 | 2 | 2 |
| 802 | 48.6 | 3 | 2 | 2 | 2 | 2 |
| 795 | 51.9 | 3 | 3 | 3 | 2 | 2 |
| 780 | 53.7 | 3 | 3 | 3 | 3 | 3 |
| 773 | 58.6 | 3 | 2 | 3 | 3 | 2 |
| Mean | 52.02 | 3.00 | 2.56 | 2.44 | 2.33 | 2.22 |
| StDev | 4.2 | 0.00 | 0.53 | 0.53 | 0.50 | 0.44 |

For the balloon up infusion, cells were administered while the balloon was inflated. The TIMI scores are shown in Table 2.

TABLE 2

| | | | Pre- | 3^{rd} | 6^{th} | |
|---|---|---|---|---|---|---|
| | Cell | | Injec- | Injec- | Injec- | |
| Animal | Dose | Pre-MI | tion | tion | tion | Final |
| ID | (×10⁶) | (TIMI) | (TIMI) | (TIMI) | (TIMI) | (TIMI) |
| 816 | 56.6 | 3 | 3 | 3 | 3 | 2 |
| 807 | 53.3 | 3 | 3 | 1 | 1 | 1 |
| 806 | 54.8 | 3 | 3 | 2 | 2 | 2 |
| 805 | 52.7 | 3 | 3 | 0 | 0 | 2 |
| 803 | 46.4 | 3 | 2 | 2 | 2 | 2 |
| 801 | 49.7 | 3 | 3 | 2 | 1 | 1 |
| 783 | 48.6 | 3 | 3 | 3 | 3 | 3 |
| 777 | 53.3 | 3 | 3 | 3 | 3 | 3 |
| 775 | 50.4 | 3 | 2 | 2 | 2 | 3 |
| Mean | 51.75 | 3.00 | 2.75 | 1.88 | 1.75 | 2.11 |
| StDev | 3.21 | 0.00 | 0.46 | 0.99 | 1.04 | 0.78 |

Cell delivery under both conditions (during balloon occlusion and without balloon occlusion) resulted in a slight drop in the TIMI flow but there was never a final TIMI flow of 0, which would be considered insufficient and likely to result in damage to heart tissue. In conclusion, the above tested doses did not result in a critical perfusion reduction as indicated by TIMI flow measured repeatedly over time. The addition of an automated and high fidelity flow or pressure measurement system would greatly improve the sensitivity and safety of this intracoronary delivery method, and would prevent the flow reduction to a TIMI grade 1 flow as observed in delivery with balloon occlusion. The system would also enable cell infusion to be increased to a higher safe level than is allowed by TIMI flow grading. TIMI flow grading is qualitative and potentially less sensitive than a quantitative flow or pressure rate measurement.

### EXAMPLE II

### Intracoronary Delivery of Cells to a Patient using a Catheter having a Flow Wire Sensor

An flow rate sensor infusion catheter of the invention (e.g., as shown in FIG. 5) is placed through a guiding catheter into the coronary artery over a guidewire. Positioning can be verified using angiography since the tip of the catheter has radio opaque markers. Blood flow in the coronary vessel is measured using the catheter's flow sensor. In addition, vasoactive agents known and used in the art, e.g., adenosine, are administered into the coronary artery to induce maximal vasodilatation, allowing measurement of maximum possible blood flow or average peak velocity without interference of vasoconstriction on an arteriolar of capillary level that would otherwise affect the measurement. Infusion of the cells is then started, while blood flow is continuously monitored. At sufficient intervals, the infusion is halted and the administration of the vasodilating agent is repeated to measure the maximum possible blood flow. During continuous cell infusion, if the blood flow in the coronary artery decreases, the infusion is stopped before a critically low blood flow that would result in tissue damage is reached.

### EXAMPLE III

### Intracoronary Delivery of Cells using a Catheter having a Flow Rate Sensor

The infusion catheter is placed through a guiding catheter into the coronary artery over a guidewire. Proper positioning can be verified using angiography since the tip of the catheter has radio opaque markers. Blood flow in the coronary vessel is automatically measured using the flow sensor. A control box continuously monitors the flow rate in the coronary artery while the cell infusion is started. The rate of infusion through the catheter is kept constant. The infusion is stopped automatically before a critical reduction in coronary blood flow is reached. Additionally, any sudden drop in coronary blood flow will cause infusion to cease and an alarm to be activated.

### EXAMPLE IV

### Intracoronary Delivery of Cells using a Catheter having a Pressure Transducer Sensor

A patient receiving an intracoronary application of regenerative cells would undergo the following exemplary procedure using an over the wire cell delivery balloon catheter with a pressure transducer sensor at its distal tip. It is understood that the procedure could vary, of course, based on the individual needs of each patient as well as the type of therapy being administered, including any adjuvant therapy. A wire would be inserted into the target coronary artery selected for infusion. The catheter would be then inserted over that wire into the target coronary artery to the affected site where regenerative cell application is desired. The balloon would be inflated with enough pressure to stop blood flow distal to it and not harm the vessel wall and/or endothelial lining. The pressure measured by the high fidelity pressure sensor would be the coronary blood pressure (similar to the systemic arterial blood pressure) before the balloon is inflated. Upon balloon inflation and complete obstruction of the blood flow the pressure that the sensor measures would drop to around 20 mmHg or whatever the capillary pressure would be at that time (the pressure can be affected by disease states i.e. myocardial infarction, cardiac hypertrophy etc.). This would be a signal to halt balloon inflation to prevent vessel wall damage.

After the balloon has been inflated, cell infusion would be started (constant flow infusion) after removal of the wire through the wire lumen or in case of a multi catheter lumen through the designated lumen. During all times, the pressure sensor would monitor the pressure distal to the balloon. As the pressure begins to rise, the cell infusion would be immediately stopped. If no rise in pressure is observed, cell infusion would continue until the desired dose of therapeutic agent has been reached. The rise in pressure could be gradual or sharp. A sharp rise in pressure can indicate a thrombotic occlusion whereas a gradual increase in pressure can indicate microvascular obstruction. After the desired amount of therapeutic agent is applied, the balloon would be deflated. At this point the pressure reading of the sensor will equal the coronary blood pressure. The catheter can then be removed from the vascular system.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A therapeutic agent delivery catheter comprising an elongated portion, a proximal end, a distal end, a guide wire, one or more lumens, and at least one sensor at the distal tip.

2. The catheter of claim 1, wherein the sensor is a flow rate sensor.

3. The catheter of claim 1, wherein the therapeutic agent is adipose derived regenerative cells.

4. The catheter of claim 1, wherein the therapeutic agent is used in intracoronary applications.

5. The catheter of claim 1, wherein the sensor is a high fidelity sensor.

6. The catheter of claim 1, wherein the distal tip is curved.

7. The catheter of claim 1, wherein the distal tip is straight.

8. The catheter of claim 2, further comprising a pressure sensor.

9. A therapeutic agent delivery catheter comprising an elongated portion, a proximal end, a distal end, a guide wire, one or more lumens, an occlusion balloon at the distal tip and a sensor distal to the occlusion balloon.

10. The catheter of claim 9, wherein the sensor is a pressure transducer sensor.

11. The catheter of claim 9, wherein the therapeutic agent is adipose derived regenerative cells.

12. The catheter of claim 9, wherein the therapeutic agent is used in intracoronary applications.

13. The catheter of claim 9, wherein the sensor is a high fidelity sensor.

14. A method of administering a therapeutic agent comprising monitoring the flow rate during infusion using the catheter of claim 2.

15. The method of claim 14, wherein administration of the therapeutic agent is halted when the flow rate decreases.

16. A method of administering a therapeutic agent comprising monitoring the pressure and flow rate using the catheter of claim 9.

17. The method of claim 16, wherein the administration of the therapeutic agent is halted when the pressure increases.
